(19) United States
(12) Patent Application Publication
(10) Pub. No.: US 2015/0231418 A1
(43) Pub. Date: Aug. 20, 2015

(54) METHOD OF CONTROLLING TISSUE TEMPERATURE AND TEMPERATURE CONTROLLING APPARATUS USING THE METHOD

(71) Applicant: SAMSUNG ELECTRONICS CO., LTD., Suwon-si (KR)

(72) Inventors: Sang-hyun KIM, Hwaseong-si (KR); Ho-taik LEE, Yongin-si (KR); Won-chul BANG, Seongnam-si (KR)

(73) Assignee: SAMSUNG ELECTRONICS CO., LTD., Suwon-si (KR)

(21) Appl. No.: 14/622,175

(22) Filed: Feb. 13, 2015

(30) Foreign Application Priority Data
Feb. 14, 2014 (KR) 10-2014-0017522

(51) Int. Cl. A61N 7/02

(52) U.S. Cl. CPC A61N 7/02

(57) ABSTRACT

A method for controlling a temperature of a tissue and a temperature controlling apparatus using the method. According to the method, a tissue parameter of a target tissue is obtained in advance and used to determine an optimum intensity of ultrasound irradiation in order to maintain the target tissue at a target temperature.

### CROSS-REFERENCE TO RELATED APPLICATIONS

This application claims priority from Korean Patent Application No. 10-2014-0017522, filed on Feb. 14, 2014, in the Korean Intellectual Property Office, the disclosure of which is incorporated herein by reference in its entirety.

### BACKGROUND

1. Field

Exemplary embodiments relate to methods for controlling a tissue temperature and temperature controlling apparatuses using the method.

2. Description of the Related Art

With advances in medicine, minimally invasive surgery has recently been replaced with noninvasive surgery for the local treatment of a tumor. An example of a non-invasive surgery method for local treatment of a tumor is a high intensity focused ultrasound (HIFU) method.

When HIFU is applied to a tissue, a tissue temperature increases due to the thermal energy of the HIFU.

### SUMMARY

Provided are methods for controlling a tissue temperature by obtaining, in advance, a tissue parameter of a target tissue, in order to reflect the characteristics of the target tissue in precisely controlling the temperature of the target tissue, and temperature controlling apparatuses using the methods.

Additional aspects will be set forth in part in the description which follows and, in part, will be apparent from the description, or may be learned by practice of the presented exemplary embodiments.

According to an aspect of one or more exemplary embodiments, a method for controlling a tissue temperature, includes: obtaining a tissue parameter with respect to a target tissue by using a degree of temperature change in the target tissue based on ultrasound irradiation; determining an intensity of ultrasound irradiation with respect to the target tissue based on a target temperature with respect to the target tissue and a measured temperature with respect to the target tissue; and applying ultrasound irradiation with the determined intensity to the target tissue.

According to another aspect of one or more exemplary embodiments, a temperature controlling apparatus includes: an ultrasound irradiator configured to irradiate ultrasound to a target tissue; a temperature measurer configured to measure a temperature of the target tissue; a parameter obtainer configured to obtain a tissue parameter with respect to the target tissue by using a degree of temperature change in the target tissue based on ultrasound irradiation; and a temperature controller configured to determine an intensity of ultrasound irradiation with respect to the target tissue based on a target temperature with respect to the target tissue and a measured temperature with respect to the target tissue, wherein the ultrasound irradiator is further configured to apply the ultrasound irradiation with the determined intensity to the target tissue.

### BRIEF DESCRIPTION OF THE DRAWINGS

These and/or other aspects will become apparent and more readily appreciated from the following description of exemplary embodiments, taken in conjunction with the accompanying drawings in which:

FIG. 1 is a block diagram which illustrates a structure of a temperature controlling apparatus, according to an exemplary embodiment;

FIG. 2 is a diagram which illustrates a temperature control unit of a temperature controlling apparatus, according to an exemplary embodiment;

FIG. 3 illustrates a method for determining a position of a target tissue to which an ultrasound irradiation is to be applied, the method being performed by an irradiation position determination module of the temperature control unit, according to an exemplary embodiment;

FIG. 4 is a graph which describes a parameter obtaining unit of a temperature controlling apparatus, according to an exemplary embodiment;

FIGS. 5A and 5B respectively show temperature distributions of a target tissue in an exemplary embodiment when temperature control with respect to a target tissue is performed without reflecting a previously obtained tissue parameter and an exemplary embodiment when temperature control with respect to a target tissue is performed by reflecting a previously obtained tissue parameter;

FIG. 6 is a flowchart which illustrates a method for controlling a tissue temperature, according to an exemplary embodiment;

FIG. 7 is a detailed flowchart which illustrates an operation of obtaining a tissue parameter with respect to a target tissue in a method for controlling a tissue temperature, according to an exemplary embodiment; and

FIG. 8 is a detailed flowchart which illustrates an operation of determining the intensity of ultrasound irradiation in a method for controlling a tissue temperature, according to an exemplary embodiment.

### DETAILED DESCRIPTION

Reference will now be made in detail to exemplary embodiments, examples of which are illustrated in the accompanying drawings, wherein like reference numerals refer to the like elements throughout. In this regard, the present exemplary embodiments may have different forms and should not be construed as being limited to the descriptions set forth herein. Accordingly, the exemplary embodiments are merely described below, by referring to the figures, to explain aspects of the present disclosure. Expressions such as “at least one of,” when preceding a list of elements, modify the entire list of elements and do not modify the individual elements of the list.

Hereinafter, the exemplary embodiments will be described in detail with reference to the attached drawings below based on examples that are just for illustration, without limiting the present inventive concept. It is intended that the exemplary embodiments are provided for purpose of description, but do not limit or define the scope of the present inventive concept. Details that are easily derivable by one of ordinary skill in the art to which the exemplary embodiments pertain based on the detailed description and exemplary embodiments are construed as being in the scope of the present disclosure.

In the present specification, the terms such as “comprise” or “include” should not be construed as necessarily including various elements or processes described in the specification, and it should be construed that some of the elements or the processes may not be included, or additional elements or processes may be further included.

In the present disclosure, terms including ordinal numbers such as ‘first’, ‘second’, etc. are used to describe various elements but the elements should not be defined by these terms. The terms are used only for distinguishing one element from another element.

The exemplary embodiments relate to a method for controlling a tissue temperature and a temperature controlling apparatus using the method. Details that are well known to one of ordinary skill in the art are omitted.

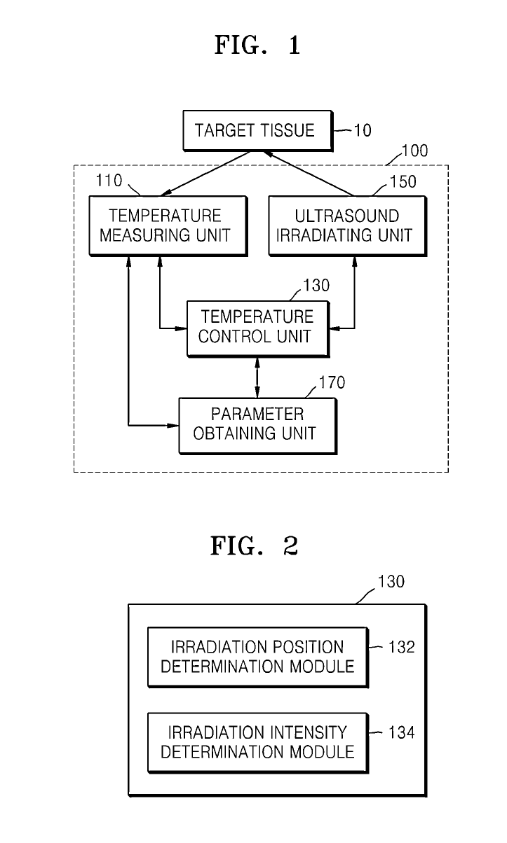

FIG. 1 is a block diagram which illustrates a structure of a temperature controlling apparatus 100, according to an exemplary embodiment. It will be apparent to one of ordinary skill in the art that the temperature controlling apparatus 100 may further include other general-use components than those illustrated in FIG. 1.

Referring to FIG. 1, the temperature controlling apparatus 100 according to an exemplary embodiment may include a temperature measuring unit (also referred to herein as a “temperature measuring device” and/or as a “temperature measurer”) 110, a temperature control unit (also referred to herein as a “temperature controller”) 130, an ultrasound irradiating unit (also referred to herein as an “ultrasound irradiation device” and/or as an “ultrasound irradiator”) 150, and a parameter obtaining unit (also referred to herein as a “parameter obtaining device” and/or as a “parameter obtainer”) 170.

The temperature measuring unit 110 measures a temperature of a target tissue 10. The temperature measuring unit 110 may measure, in real-time, a temperature of the entire target tissue 10. The target tissue 10 may be a living tissue which includes a lesion, such as a tumor. The temperature measuring unit 110 may be a temperature measuring unit that is insertable into a body to measure a temperature of the target tissue 10, such as a thermocouple, or a unit that is capable of measuring a temperature of the target tissue 10 via a non-contact imaging method, such as, for example, a ultrasound thermometry. To control a temperature of a target tissue through ultrasound irradiation, the temperature measuring unit 110 may first measure a temperature change in a target tissue based on ultrasound irradiation for a short period of time. Further, the temperature measuring unit 110 may continuously measure a temperature change in a target tissue while controlling a temperature of the target tissue via ultrasound irradiation.

The temperature control unit 130 determines an intensity of ultrasound to be irradiated to the target tissue 10 based on a target temperature with respect to the target tissue 10 and a temperature of the target tissue 10 which is measured by using the temperature measuring unit 110. In particular, the temperature control unit 130 may determine an optimum intensity of ultrasound irradiation for each predetermined unit time so that the target tissue 10 is maintained at a predetermined temperature for treatment purposes. The temperature controlling apparatus 100 includes the temperature control unit 130 for adjusting via thermal energy caused by ultrasound irradiation the temperature of the target tissue 10 to reach a target temperature at which treatment is optimized. The target temperature may vary based on a treatment method with respect to the target tissue 10. For example, when a lesion of the target tissue 10 is treated by using hyperthermia, a temperature at which a living tissue is safe may be set as a target temperature in order to prevent destruction of the target tissue 10.

The ultrasound irradiating unit 150 irradiates ultrasound to the target tissue 10. The ultrasound irradiating unit 150 may irradiate ultrasound at a predetermined intensity. The ultrasound irradiating unit 150 may include, for example, a transducer that generates and irradiates ultrasound and a driver that drives the transducer. When the intensity of ultrasound irradiation which is determined by using the temperature control unit 130 is input to the driver of the ultrasound irradiating unit 150, ultrasound to be irradiated to the target tissue 10 may be generated by using the transducer. Further, when information about a position to which ultrasound is to be irradiated is input to the driver of the ultrasound irradiating unit 150 by using the temperature control unit 130, ultrasound may also be irradiated to a desired position on the target tissue 10.

The temperature control unit 130 of the temperature controlling apparatus 100 may determine an optimum intensity of ultrasound irradiation by further reflecting the characteristics of the target tissue 10 when determining the intensity of ultrasound irradiation with respect to the target tissue 10 in order to precisely control a temperature of the target tissue 10. In particular, the temperature control unit 130 may determine an optimum intensity of ultrasound irradiation by using a tissue parameter which indicates the characteristics of a tissue in regard to determining the optimum intensity with respect to the target tissue 10. To this end, the temperature controlling apparatus 100 includes the parameter obtaining unit 170.

The parameter obtaining unit 170 obtains a tissue parameter with respect to the target tissue 10 by using a degree of temperature change in the target tissue 10 based on ultrasound irradiation. In particular, in order to control a temperature of the target tissue 10 through ultrasound irradiation, the parameter obtaining unit 170 may first obtain a degree of temperature change in the target tissue 10 according to time after ultrasound is irradiated for a short period of time, and then may estimate a tissue parameter based on the obtained degree of temperature change to thereby obtain a tissue parameter. For example, by adjusting within a predetermined range a tissue parameter used in a bio heat transfer model with respect to the target tissue 10, the parameter obtaining unit 170 may estimate a tissue parameter in a bio heat transfer model that mostly corresponds to the obtained degree of temperature change as a tissue parameter with respect to the target tissue 10.

The temperature control unit 130 and the ultrasound irradiating unit 150 may repeatedly operate such that a temperature measured by using the temperature measuring unit 110 reaches the target temperature with respect to the target tissue 10. In particular, the temperature control unit 130 and the ultrasound irradiating unit 150 may determine and irradiate ultrasound with an optimum intensity for each predetermined unit time such that the target tissue 10 reaches a predetermined temperature for treatment, and after the target tissue 10 has reached the target temperature, the temperature control unit 130 and the ultrasound irradiating unit 150 may determine an optimum intensity for ultrasound irradiation for each predetermined unit time and irradiate corresponding ultrasound in order to maintain the target temperature. The predetermined unit time may be within a range of between several milliseconds and several hundreds of milliseconds or less.

Hereinafter, the temperature control unit 130 will be described in detail with reference to FIG. 2.

FIG. 2 is a diagram which illustrates the temperature control unit 130 of the temperature controlling apparatus (100), according to an exemplary embodiment. It will be apparent to one of ordinary skill in the art that other general-use components than the components illustrated in FIG. 2 may be further included in the temperature control unit 130.

The temperature control unit 130 may determine an intensity of ultrasound irradiation by applying, to a bio heat transfer model, a difference between a target temperature with respect to the target tissue 10 and a temperature of the target tissue 10 which is measured by using the temperature measuring unit 110. As illustrated in FIG. 2, the temperature control unit 130 may include an irradiation position determination module 132 and an irradiation intensity determination module 134.

The irradiation position determination module 132 determines a position of a portion of the target tissue 10 to which ultrasound is to be irradiated. The irradiation position determination module 132 may determine a position of a portion of the target tissue 10 to which ultrasound is to be irradiated for each predetermined unit time so that the entire area of the target tissue 10 is maintained at a target temperature. In the case of high intensity focused ultrasound (HIFU), ultrasound energy may not be simultaneously irradiated over the entire area of the target tissue 10 to increase a temperature of the target tissue 10. Thus, ultrasound energy may be irradiated by changing portions of the target tissue 10 for irradiation for each predetermined unit time. A similar effect as if ultrasound energy is irradiated over the entire area of the target tissue 10 may be obtained when each predetermined unit time for irradiating ultrasound energy to a determined position of a portion of the target tissue 10 is very short.

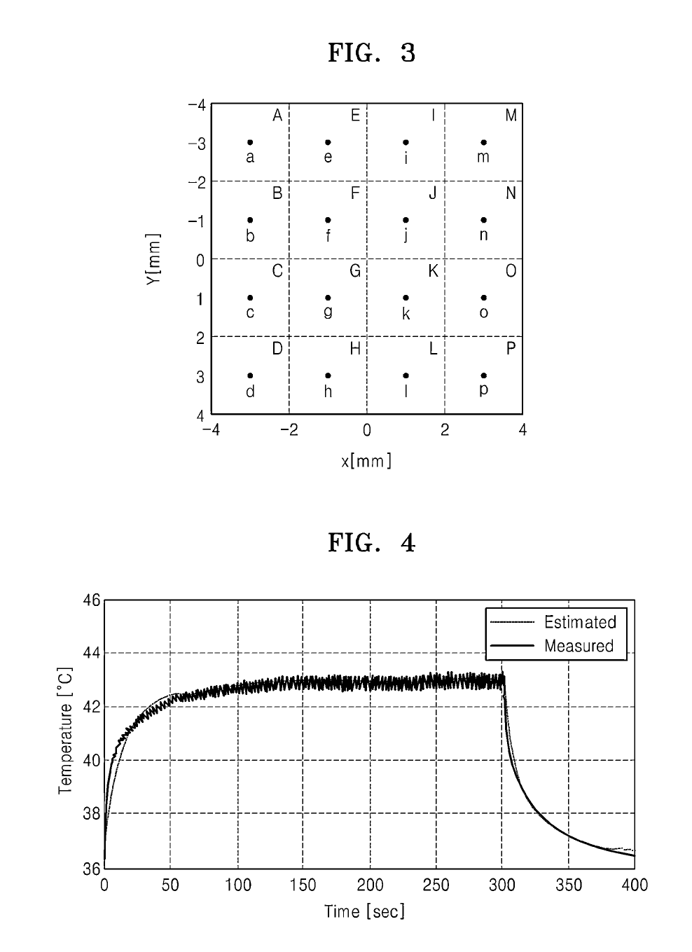

The irradiation position determination module 132 may determine at least one position of a portion of the target tissue 10 to which an ultrasound irradiation is to be applied for each predetermined unit time. In particular, the irradiation position determination module 132 may determine one point or at least two points within the target tissue 10 for each predetermined unit time as a position to which an ultrasound irradiation is to be applied. For example, the ultrasound irradiating unit 150 may determine a plurality of foci where ultrasound energy is focused with respect to the target tissue 10, and the irradiation position determination module 132 may determine at least one of the plurality of foci as a position where ultrasound energy is to be irradiated. Hereinafter, a method of determining a position of a portion of the target tissue 10 to which ultrasound energy is to be irradiated by using the irradiation position determination module 132 will be described with reference to FIG. 3.

FIG. 3 illustrates a method for determining a position of a portion of a target tissue where ultrasound energy is to be irradiated, the method being performed by using the irradiation position determination module 132 of the temperature control unit 130, according to an exemplary embodiment.

Referring to FIG. 3, a target tissue 10 having a surface area of 8 mm×8 mm is illustrated. As illustrated in FIG. 3, the target tissue 10 may be divided into sixteen equal sub-areas, and ultrasound energy may be focused on a center of each sub-area. In particular, the target tissue 10 may be divided into a total of sixteen sub-areas, each of which has an area of 2 mm×2 mm, that is, sub-areas A to P, and a total of sixteen foci, that is foci a through p, may be respectively formed at centers of the respective sub-areas. Each sub-area is affected by ultrasound that is focused on the focus located at the center of each sub-area. In this aspect, when ultrasound energy is focused on focus a, sub-area A mainly receives thermal energy due to the ultrasound energy, and a temperature of sub-area A increases accordingly.

The irradiation position determination module 132 of the temperature control unit 130 may determine at least one of positions of a plurality of foci where ultrasound energy is focused on the target tissue 10 as a position where an ultrasound irradiation is to be applied.

The irradiation position determination module 132 may determine a position where ultrasound energy is to be irradiated in consideration of temperatures at the respective positions of the plurality of foci. For example, the irradiation position determination module 132 may determine a position of focus having a lowest temperature from among temperatures of positions of the plurality of foci, as an ultrasound irradiation position. In particular, when a target temperature is 42° C., temperatures of foci a through o are each 40° C., and a temperature of focus p is 38° C., focus p may be determined as an ultrasound irradiation position.

The irradiation position determination module 132 may also determine an ultrasound irradiation position by considering an average temperature of the target tissue 10 that is obtained by considering weights for respective distances from the positions of the plurality of respective foci on the target tissue 10 to an arbitrary position on the target tissue 10. For example, the irradiation position determination module 132 may calculate an average temperature of the target tissue 10 by applying a lower weight as a distance from the respective positions of the foci on the target tissue 10 to an arbitrary position on the target tissue 10 increases, and may determine an ultrasound irradiation position by considering the average temperature of the target tissue 10. The irradiation position determination module 132 may also determine an ultrasound irradiation position by applying a weight of zero to other positions where no focus exists, and calculating an average temperature of each area by applying a weight based on distance only to a position within an area of the target tissue 10 where each focus is included. In particular, for focus a, an ultrasound irradiation position may be determined with respect to a weighted average temperature that is calculated by applying a weight according to a distance between focus a and an arbitrary position within sub-area A. Further, for foci b through p, an ultrasound irradiation position may be determined with respect to each weighted average temperature that is calculated in the above-described manner.

The irradiation position determination module 132 may determine at least two foci at the same time as ultrasound irradiation positions. In particular, according to the above-described method, when at least two foci have the same value, all of the at least two foci may be determined as ultrasound irradiation positions. For example, from among foci a through p, if temperatures or average temperatures of focus f and focus k are the lowest, both focus f and focus k may be determined as ultrasound irradiation positions. Moreover, when a first priority focus and a second priority focus are determined according to the above-described method, both the first priority focus and the second priority focus may be determined as ultrasound irradiation positions. For example, when a temperature or an average temperature of focus g from among foci a through p is the lowest, and a temperature or an average temperature of focus j is next to the lowest, both focus g and focus j may be determined as ultrasound irradiation positions.

Referring to FIG. 2 again, the irradiation intensity determination module 134 determines an intensity of ultrasound irradiation with respect to the determined position based on a target temperature with respect to the target tissue 10 and a temperature of a portion of the target tissue 10 which is measured with respect to the position determined by using the irradiation position determination module 132. In this aspect, an intensity of ultrasound irradiation with respect to the position which is determined by using the irradiation position determination module 132 may be determined by using a bio heat transfer model.

A bio heat transfer model is a mathematical expression of a change in a temperature of a predetermined tissue, and may be expressed as in Equation 1 below.

$${ρ}_{t} \cdot {C}_{t} \cdot \frac{\partial T ( x , t )}{\partial t} = {k}_{1} \cdot {∇}^{2} T ( x , t ) + {V}_{{ρ}_{b}} \cdot {C}_{b} \cdot ( {T}_{b} - T ( x , t ) ) + Q ( x , t ) , \qquad [ Equation 1 ]$$

where ρₜ denotes a density of a tissue, Cₜ is a specific heat of the tissue. T(x,t) is a temperature at a position x in a tissue and at a time t,

$$\frac{\partial T ( x , t )}{\partial t}$$

denotes a variation of T(x,t) with respect to time t and indicates a primary differential value with respect to a time t, and ∇²T(x,t) denotes a variation with respect to a position T(x,t) and indicates a secondary spatial differential value with respect to a position x. kₜ denotes a thermal conductivity of a tissue, V_{ρ}_{_{b}} denotes a perfusion rate of hematocele in a tissue, C_{b} is a specific heat of hematocele in a tissue, T_{b}, denotes a temperature of hematocele in a tissue. Q(x,t) denotes heat applied from the outside to a position x in a tissue at a time t.

Heat (Q(x,t)) that is applied from the outside to a position x in a tissue at a time t may be generated by using any one or more of various heat generators. For example, heat may be applied to a tissue by irradiating ultrasound energy. In this case, heat (Q) that is applied to a tissue by irradiating ultrasound energy thereto may be expressed as in Equation 2 below.

Q=2·β·f·I [Equation 2]

where β denotes an absorption coefficient of a tissue, f denotes a frequency of the ultrasound, and I denotes an intensity of ultrasound irradiation.

When heat is applied to a tissue by irradiating ultrasound thereto, an intensity of ultrasound irradiation may be expressed as in Equation 3 below by using Equations 1 and 2.

$$I = \frac{\begin{matrix}{ρ}_{t} \cdot {C}_{t} \cdot \frac{\partial T ( x , t )}{\partial t} - {k}_{t} \cdot {∇}^{2} T ( x , t ) - \\ {V}_{{ρ}_{b}} \cdot {C}_{b} \cdot ( {T}_{b} - T ( x , t ) )\end{matrix}}{2 \cdot β \cdot f \cdot 10} \qquad [ Equation 3 ]$$

The temperature controlling apparatus 100 according to the current exemplary embodiment may determine an intensity of ultrasound irradiation at which a target temperature may be reached by applying as a difference between a target temperature and a measured temperature a value

$$\frac{\partial T ( x , t )}{\partial t}$$

which denotes a primary differential value at a time t as a variation with respect to a time T(x,t) as expressed in Equation 3. For precise temperature control with respect to the target tissue 10, the temperature control unit 130 may determine an optimum intensity of ultrasound irradiation by using a tissue parameter which represents tissue characteristics of the target tissue 10. In particular, the temperature control unit 130 may determine an intensity of ultrasound irradiation by applying a difference between a target temperature and a measured temperature of the target tissue 10 to a bio heat transfer model with respect to which a tissue parameter which is obtained by using the parameter measuring unit 170 is used. To this end, the parameter obtaining unit 170 may obtain, in advance, at least one of a perfusion rate in hematocele in a tissue, a thermal conductivity of a tissue, and an absorption coefficient of a tissue from among tissue parameters used in a bio heat transfer model.

When there are at least two positions to which ultrasound energy is to be irradiated, the irradiation intensity determination module 134 may determine an intensity of ultrasound irradiation with respect to determined positions based on a temperature that is closest to a target temperature from among temperatures measured with respect to the determined positions. For example, when a target temperature is 42° C., temperatures of foci a through n are all 40° C., a temperature of focus o is 37° C., and a temperature of focus p is 38° C., 38° C., which is the temperature of focus p, may be used when determining an intensity of ultrasound irradiation with respect to focus n and focus o. When two positions where ultrasound energy is to be irradiated are determined by using the irradiation position determination module 132, and temperatures or average temperatures at the two positions are the same, a single, measured temperature is to be applied to a bio heat transfer model, and thus, the single, measured temperature may be applied. However, when a first priority position and a second priority position are both determined as positions where ultrasound energy is to be irradiated, an intensity of ultrasound irradiation may be determined by applying a temperature or an average temperature of the second priority position as a measured temperature used in a bio heat transfer model. Thus, a target temperature at any one of the two positions may be prevented from reaching a higher value even if ultrasound energy of the determined irradiation intensity is irradiated to the two positions. If the target temperature is higher than the target temperature, the target tissue 10 might be destroyed.

Alternatively, when there are at least two positions where ultrasound is to be irradiated, the irradiation intensity determination module 134 may determine an intensity of ultrasound irradiation with respect to each of the two positions based on temperatures which are respectively measured regarding the determined positions. For example, in the above example, for focus o, an intensity of ultrasound irradiation may be determined based on the value of 37° C. which is a measured temperature at focus o, and for focus p, an intensity of ultrasound irradiation may be determined based on a value of 38° C. which is a measured temperature at focus p.

Alternatively, with respect to the irradiation intensity determination module 134, when there are at least two positions where ultrasound is to be irradiated, the irradiation intensity determination module 134 may determine an intensity of ultrasound irradiation within a safe range with respect to each of the determined positions based on a temperature that is most different from the target temperature from among temperatures respectively measured with respect to the determined positions. In the above example, an intensity of ultrasound irradiation may be determined based on a value of 37° C., which is a temperature of focus n. However, the intensity of ultrasound irradiation that is determined in this case should be within a safe range with respect to each of the determined positions.

In addition, the irradiation position determination module 132 may also determine an ultrasound irradiation position by using a bio heat transfer model. The irradiation position determination module 132 may determine an ultrasound irradiation position by predicting a temperature distribution of the target tissue 10 by applying a temperature which is measured with respect to the target tissue 10 and ultrasound with a predetermined irradiation intensity to a bio heat transfer model. In particular, when ultrasound is focused on a predetermined position of the target tissue 10, heat is transferred in the target tissue 10 through heat diffusion, and the irradiation position determination module 132 may predict a temperature distribution of the target tissue 10 and also determine an optimum position by using a bio heat transfer model in consideration of the heat diffusion. However, in this case, for precise temperature control, the irradiation position determination module 132 may also determine an optimum ultrasound irradiation position by using a tissue parameter which represents characteristics of a tissue when determining an ultrasound irradiation position with respect to the target tissue 10. In this aspect, the irradiation position determination module 132 may determine an ultrasound irradiation position by predicting a temperature distribution of the target tissue 10 by applying a temperature which is measured with respect to the target tissue 10 and ultrasound with a predetermined irradiation intensity to a bio heat transfer model by using a tissue parameter obtained by using the parameter measuring unit 170. To this end, the parameter obtaining unit 170 may obtain, in advance, at least one of a perfusion rate in hematocele in a tissue, a thermal conductivity of a tissue, and an absorption coefficient of a tissue from among tissue parameters used in a bio heat transfer model.

An ultrasound irradiation position determined by using the irradiation position determination module 132 and an intensity of ultrasound irradiation determined by using the irradiation intensity determination module 134 are transferred to the ultrasound irradiating unit 150, and the ultrasound irradiating unit 150 may generate an ultrasound irradiation having the determined intensity and irradiate the same to the determined ultrasound irradiation position.

FIG. 4 is a graph which describes the parameter obtaining unit 170 of the temperature controlling apparatus (100), according to an exemplary embodiment.

As described above, the irradiation position determination module 132 or the irradiation intensity determination module 134 may use a bio heat transfer model in determining an ultrasound irradiation position or an intensity of ultrasound irradiation. For precise temperature control with respect to the target tissue 10, tissue parameters used in a bio heat transfer model are obtained in advance.

The temperature controlling apparatus 100, according to the current exemplary embodiment, may include the parameter obtaining unit 170, and may obtain, in advance, tissue parameters used in a bio heat transfer model by using the parameter obtaining unit 170.

The parameter obtaining unit 170 may determine a degree of temperature change in the target tissue 10 as a function of time after ultrasound is irradiated to the target tissue 10, and may obtain a tissue parameter by estimating a tissue parameter based on the determined degree of temperature change. In detail, the parameter obtaining unit 170 may adjust, within a predetermined range, a tissue parameter used in a bio heat transfer model so as to estimate a tissue parameter that corresponds to the determined degree of temperature change as a tissue parameter with respect to the target tissue 10.

Referring to FIG. 4, the degree of temperature change which is determined by using the parameter obtaining unit 170 may be confirmed by using a temperature value measured by using the temperature measuring unit 110. In the degree of temperature change, a change in temperatures measured for 400 seconds with respect to the target tissue 10 is shown. As shown in FIG. 4, in a first section, the temperature rapidly increases due to ultrasound irradiation, in a second section the temperature remains relatively constant, and after about 300 seconds, in a third section, the temperature decreases as ultrasound irradiation is stopped.

The parameter obtaining unit 170 may estimate a tissue parameter that almost matches a graph of a degree of temperature change by adjusting a value of a tissue parameter used in a bio heat transfer model with respect to the target tissue 10 based on the degree of temperature change which is determined by using a temperature value measured by using the temperature measuring unit 110. Referring to FIG. 4, a graph that almost corresponds to a degree of temperature change indicating a temperature of the target tissue 10 that is actually measured for a predetermined period of time according to a bio heat transfer model is generated by adjusting a tissue parameter used in the bio heat transfer model. The parameter obtaining unit 170 may estimate the above tissue parameter as a tissue parameter with respect to the target tissue 10. A tissue parameter that the parameter obtaining unit 170 may determine may include at least one of a perfusion rate in hematocele in a tissue, a thermal conductivity of a tissue, and an absorption coefficient of a tissue from among tissue parameters used in the bio heat transfer model. A tissue parameter that is determined by using the parameter obtaining unit 170 may be transmitted to the temperature control unit 130.

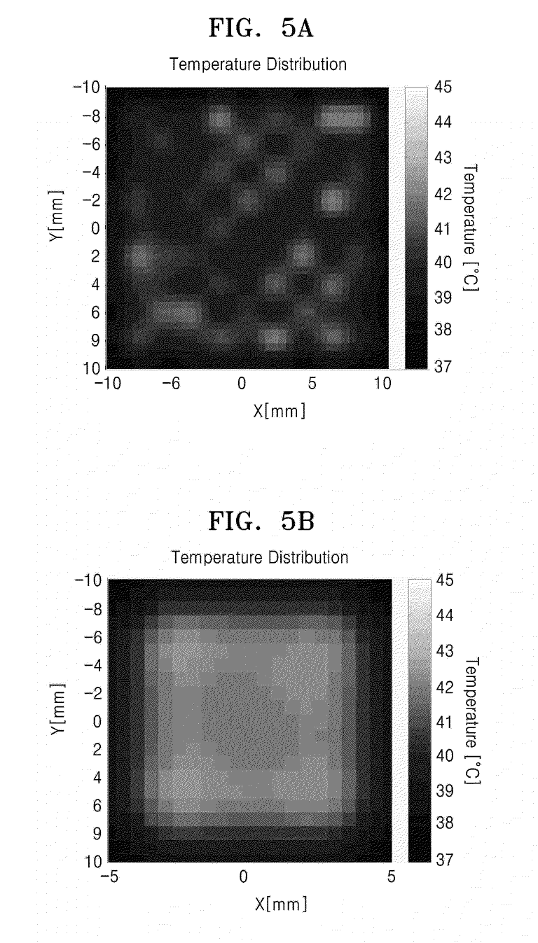

FIGS. 5A and 5B respectively show temperature distributions of a target tissue with respect to an exemplary embodiment in which a temperature control with respect to a target tissue is performed without taking into account a previously obtained tissue parameter and with respect to an exemplary embodiment in which a temperature control with respect to a target tissue is performed by taking into account a previously obtained tissue parameter.

Compared to FIG. 5A showing a temperature distribution with respect to the target tissue 10 when a temperature control with respect to the target tissue 10 is performed without taking into account a previously obtained tissue parameter, an area having a target temperature of 42° C. is significantly broader in the graph of FIG. 5B which indicates a temperature distribution with respect to the target tissue 10 when a temperature control with respect to the target tissue 10 is performed by taking into account a previously obtained tissue parameter. In particular, when a temperature control with respect to the target tissue 10 is performed by taking into account a tissue parameter with respect to the target tissue 10, precise temperature control may be performed.

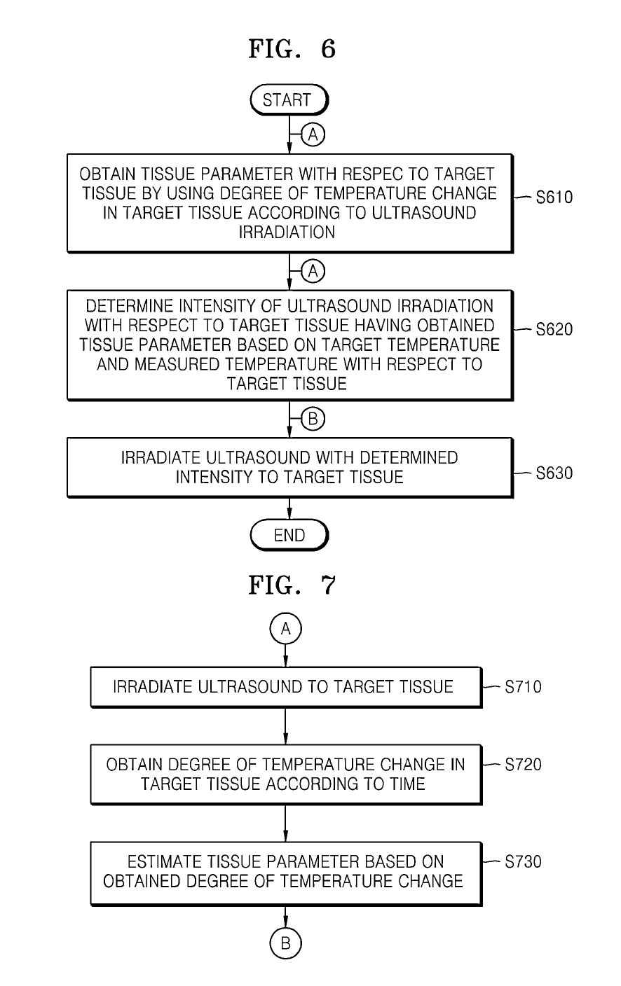

FIG. 6 is a flowchart which illustrates a method for controlling a tissue temperature, according to an exemplary embodiment. The above descriptions of the temperature controlling apparatus 100 according to the exemplary embodiments are also included in the current exemplary embodiment.

In operation S610, the parameter obtaining unit 170 obtains a tissue parameter with respect to the target tissue 10 by using a degree of temperature change in the target tissue 10 based on ultrasound irradiation. The temperature control unit 130 of the temperature controlling apparatus 100 irradiates ultrasound having an optimum intensity based on an ultrasound irradiation position or an irradiation intensity with respect to the target tissue 10. Hereinafter, an operation of obtaining a tissue parameter with respect to the target tissue 10 will be described in detail with reference to FIG. 7.

FIG. 7 is a detailed flowchart which illustrates an operation of obtaining a tissue parameter with respect to the target tissue 10 in a method for controlling a tissue temperature, according to an exemplary embodiment.

In operation S710, the ultrasound irradiating unit 150 irradiates ultrasound to the target tissue 10 in order to obtain a degree of temperature change in the target tissue 10 which is to be used to obtain a tissue parameter with respect to the target tissue 10 in advance. In particular, heat is generated in the target tissue 10 for a short period of time.

In operation S720, the parameter obtaining unit 170 determines a degree of temperature change in the target tissue 10 as a function of time. In detail, the parameter obtaining unit 170 may determine a degree of temperature change in the target tissue 10 based on a temperature of the target tissue 10 that is measured by using the temperature measuring unit 110. A degree of temperature change in the target tissue 10 may include a first section where the temperature increases, a second section where the temperature is relatively constant, and a third section where the temperature decreases, or the like, and an actual reaction of the target tissue 10 to heat may be observed from the degree of temperature change in the target tissue 10.

In operation S730, the parameter obtaining unit 170 estimates a tissue parameter from the determined degree of temperature change. The parameter obtaining unit 170 may estimate a tissue parameter that corresponds to the determined degree of temperature change as a tissue parameter with respect to the target tissue 10 by adjusting a tissue parameter used in a bio heat transfer model with respect to the target tissue 10 within a predetermined range. By adjusting the tissue parameter used in the bio heat transfer model, the parameter obtaining unit 170 may estimate a tissue parameter as a tissue parameter with respect to the target tissue 10 when a temperature change according to the bio heat transfer model almost corresponds to a degree of temperature change indicating a temperature of the target tissue 10 that is actually measured for a predetermined period of time. A tissue parameter that the parameter obtaining unit 170 may determine may include at least one of a perfusion rate in hematocele in a tissue, a thermal conductivity of a tissue, and an absorption coefficient of a tissue from among tissue parameters used in a bio heat transfer model.

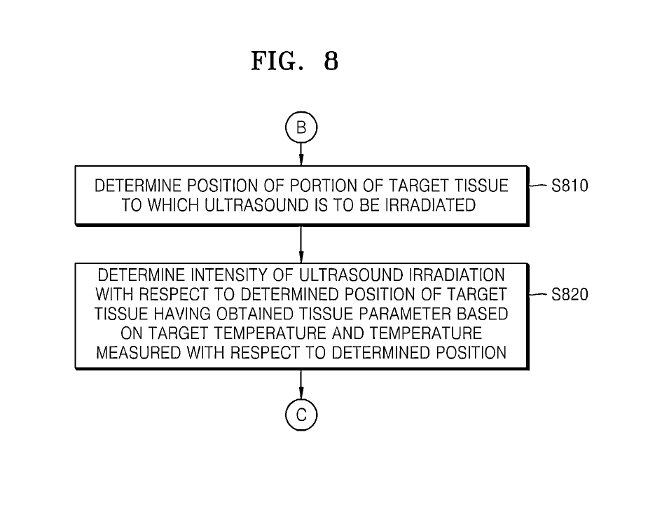

Referring to FIG. 6 again, in operation S620, the temperature control unit 130 determines, based on a target temperature and a measured temperature with respect to the target tissue 10, an intensity of ultrasound irradiation with respect to the target tissue 10 having a tissue parameter that is determined by using the parameter obtaining unit 170. The intensity of ultrasound irradiation may be determined by applying a difference between a target temperature and a measured temperature with respect to the target tissue 10 to a bio heat transfer model using a tissue parameter obtained via the parameter obtaining unit 170. Hereinafter, an operation of determining the intensity of ultrasound irradiation will be described in detail with reference to FIG. 8.

FIG. 8 is a detailed flowchart which illustrates an operation of determining an intensity of ultrasound irradiation in a method for controlling a tissue temperature, according to an exemplary embodiment.

In operation S810, the irradiation position determination module 132 of the temperature control unit 130 determines a position of a portion of the target tissue 10 to which ultrasound energy is to be irradiated. The irradiation position determination module 132 may determine at least one position of a portion of the target tissue 10 to which ultrasound energy is to be irradiated for each predetermined unit time. For example, the irradiation position determination module 132 may determine at least one of positions of a plurality of foci as an ultrasound irradiation position in consideration of temperatures of the positions of the respective foci on which ultrasound is focused with respect to the target tissue 10. In addition, alternatively, the irradiation position determination module 132 may determine an ultrasound irradiation position by predicting a temperature distribution of the target tissue 10 by applying a temperature which is measured with respect to the target tissue 10 and a predetermined irradiation intensity to a bio heat transfer model using a tissue parameter which is determined by using the parameter obtaining unit 170.

In operation S820, the irradiation intensity determination module 134 of the temperature control unit 130 determines an intensity of ultrasound irradiation with respect to a position of the target tissue 10 having the tissue parameter that is determined by using the parameter obtaining unit 170, and the position is determined based on a target temperature and a temperature which is measured with respect to the position determined by using the irradiation position determination module 132. According to the method for controlling a temperature according to the exemplary embodiments, an intensity of ultrasound irradiation with respect to a position determined by using the irradiation position determination module 132 may be determined by applying a difference between a target temperature and a measured temperature to a bio heat transfer model using a tissue parameter which is determined by using the parameter obtaining unit 170. Temperature control with respect to the target tissue 10 may be accurately performed by using the tissue parameter that is obtained in advance with respect to the target tissue 10 in temperature control of the target tissue 10.

Referring to FIG. 6 again, in operation S630, the ultrasound irradiating unit 150 irradiates, to the target tissue 10, ultrasound energy with an intensity determined by using the irradiation intensity determination module 134 of the temperature control unit 130. An ultrasound irradiation position determined by using the irradiation position determination module 132 of the temperature control unit 130 and an intensity of ultrasound irradiation determined by using the irradiation intensity determination module 134 of the temperature control unit 130 are transmitted to the ultrasound irradiating unit 150. By using the ultrasound irradiation position and the intensity of ultrasound irradiation, the ultrasound irradiating unit 150 may generate ultrasound energy which has the determined intensity, and irradiate the ultrasound energy to the determined ultrasound irradiation position.

Operation S620 of determining the intensity of ultrasound irradiation and operation S630 of irradiating ultrasound energy to the target tissue 10 may be repeated for each predetermined unit time such that the measured temperature reaches a target temperature with respect to the target tissue 10.

As described above, according to the one or more of the above-described exemplary embodiments, a tissue parameter of a target tissue is obtained in advance, and the obtained tissue parameter is used in determining an optimum intensity of ultrasound irradiation in order to maintain the target tissue at a target temperature, thereby precisely controlling a temperature of the target tissue.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope of the present inventive concept as defined by the following claims.

What is claimed is:

1. A method for controlling a tissue temperature, the method comprising:
obtaining a tissue parameter with respect to a target tissue by using a degree of temperature change in the target tissue based on ultrasound irradiation;
determining an intensity of ultrasound irradiation with respect to the target tissue based on a target temperature with respect to the target tissue and a measured temperature with respect to the target tissue; and
applying ultrasound irradiation with the determined intensity to the target tissue.

2. The method of claim 1, wherein the obtaining the tissue parameter comprises:
applying ultrasound irradiation to the target tissue;
determining a degree of temperature change in the target tissue as a function of time; and
estimating the tissue parameter based on the determined degree of temperature change.

3. The method of claim 2, wherein in the estimating the tissue parameter, a predetermined tissue parameter which is used in a bio heat transfer model with respect to the target tissue is adjusted within a predetermined range so as to estimate a tissue parameter that corresponds to the determined degree of temperature change in the target tissue as being the tissue parameter with respect to the target tissue.

4. The method of claim 2, wherein the tissue parameter includes at least one from among a perfusion rate of hematocele in a tissue, a thermal conductivity of a tissue, and an absorption coefficient of a tissue.

5. The method of claim 2, wherein the degree of temperature change in the target tissue comprises a first time interval during which a temperature increases, a second time interval during which the temperature is constant, and a third time interval during which the temperature decreases.

6. The method of claim 1, wherein in the determining the intensity of the ultrasound irradiation, a difference between the target temperature and the measured temperature is applied to a bio heat transfer model using the obtained tissue parameter.

7. The method of claim 1, further comprising determining a position of a portion of the target tissue to which the ultrasound irradiation is to be applied,
wherein in the determining the intensity of the ultrasound irradiation, an intensity of the ultrasound irradiation with respect to the determined position is determined based on the target temperature and the measured temperature with respect to the determined position.

8. The method of claim 7, wherein the determining the position includes predicting a temperature distribution of the target tissue by applying the measured temperature and an ultrasound irradiation of a predetermined intensity to a bio heat transfer model using the obtained tissue parameter.

9. The method of claim 7, wherein the determining the position includes determining the position from among a plurality of positions which correspond to a plurality of foci on which the ultrasound irradiation is focused.

10. The method of claim 1, wherein the determining the intensity of the ultrasound irradiation and the applying the ultrasound irradiation to the target tissue are repeatedly performed until the measured temperature reaches the target temperature with respect to the target tissue.

11. A temperature controlling apparatus comprising:
an ultrasound irradiator configured to irradiate ultrasound to a target tissue;
a temperature measurer configured to measure a temperature of the target tissue;
a parameter obtainer configured to obtain a tissue parameter with respect to the target tissue by using a degree of temperature change in the target tissue based on ultrasound irradiation; and
a temperature controller configured to determine an intensity of ultrasound irradiation with respect to the target tissue based on a target temperature with respect to the target tissue and a measured temperature with respect to the target tissue,
wherein the ultrasound irradiator is further configured to apply the ultrasound irradiation with the determined intensity to the target tissue.

12. The temperature controlling apparatus of claim 11, wherein the parameter obtainer is further configured to determine a degree of temperature change in the target tissue as a function of time after the ultrasound irradiation has been applied, and to estimate the tissue parameter based on the determined degree of temperature change.

13. The temperature controlling apparatus of claim 12, wherein the parameter obtainer is further configured to estimate the tissue parameter which corresponds to the determined degree of temperature change by adjusting, within a predetermined range, a predetermined tissue parameter which is used in a bio heat transfer model with respect to the target tissue.

14. The temperature controlling apparatus of claim 12, wherein the tissue parameter includes at least one from among a perfusion rate of hematocele in a tissue, a thermal conductivity of a tissue, and an absorption coefficient of a tissue.

15. The temperature controlling apparatus of claim 12, wherein the degree of temperature change comprises a first time interval during which a temperature increases, a second time interval during which the temperature is constant, and a third time interval during which the temperature decreases.

16. The temperature controlling apparatus of claim 11, wherein the temperature controller is further configured to determine the intensity of the ultrasound irradiation by applying a difference between the target temperature and the measured temperature to a bio heat transfer model.

17. The temperature controlling apparatus of claim 11, wherein the temperature controller comprises:
an irradiation position determination module configured to determine a position of a portion of the target tissue to which the ultrasound irradiation is to be applied; and
an irradiation intensity determination module configured to determine an intensity of the ultrasound irradiation with respect to the determined position based on the target temperature and the measured temperature with respect to the determined position.

18. The temperature controlling apparatus of claim 17, wherein the irradiation position determination module is further configured to determine the position by predicting a temperature distribution of the target tissue by applying the measured temperature and an ultrasound irradiation of a predetermined intensity to a bio heat transfer model using the obtained tissue parameter.

19. The temperature controlling apparatus of claim 17, wherein the irradiation position determination module is further configured to determine the position from among a plurality of positions which correspond to a plurality of foci on which the ultrasound irradiation is focused.

20. The temperature controlling apparatus of claim 11, wherein the temperature controller and the ultrasound irradiator are further configured to repeatedly operate until the measured temperature reaches the target temperature with respect to the target tissue.
